(19) United States
(12) Patent Application Publication
(10) Pub. No.: US 2019/0218598 A1
(43) Pub. Date: Jul. 18, 2019

(54) GRAPHENE NANOSENSOR FOR DETECTING TARGET RNA

(71) Applicant: SEOUL NATIONAL UNIVERSITY R&DB FOUNDATION, Seoul (KR)

(72) Inventors: Do Won HWANG, Gyeonggi-do (KR); Dong Soo LEE, Seoul (KR); Yoo Ri CHOI, Seoul (KR); Mi Young KIM, Seoul (KR)

(21) Appl. No.: 16/324,467

(22) PCT Filed: Aug. 10, 2017
(86) PCT No.: PCT/KR2017/008687

§ 371 Date: Feb. 8, 2019

(30) Foreign Application Priority Data
Aug. 10, 2016 (KR) 10-2016-0101592

(51) Int. Cl. C12Q 1/6825; C01B 32/23

(52) U.S. Cl. CPC C12Q 1/6825; C12Q 2600/158; C01B 32/23

(57) ABSTRACT

A graphene nanosensor is capable of: simply, quickly and accurately detecting RNA biomarkers that have disease-specific over-expression, as well as an expression level thereof, in living tissues or cells; obtaining a product with high reliability and resolution. The graphene nanosensor enables rapid diagnosis of a disease and being helpful for establishing treatment policy of the disease. The graphene nanosensor may rapidly and simply detect a target RNS with high sensitivity at low costs, thereby expecting superior effects when used in clinical applications and thus replacing the FISH method.

### CROSS REFERENCE TO RELATED APPLICATIONS AND CLAIM OF PRIORITY

This application claims benefit under 35 U.S.C. 119(e), 120, 121, or 365(c), and is a National Stage entry from International Application No. PCT/KR2017/008687, filed Aug. 10, 2017, which claims priority to the benefit of Korean Patent Application No. 10-2016-0101592 filed in the Korean Intellectual Property Office on Aug. 10, 2016, the entire contents of which are incorporated herein by reference.

### TECHNICAL FIELD

The present invention relates to a graphene nanosensor for detecting a RNA biomarker in tissues or cells.

### BACKGROUND ART

Modern technologies widely used in personalized medicine and disease diagnosis are based on biomarker detection. A biomarker mostly consists of protein or nucleic acid, and executes immunostaining with respect to a tissue obtained for detection of biomarkers, or in a case of the nucleic acid, a fluorescence in situ hybridization (FISH) analysis with targeting the nucleic acid.

Recently, RNA markers including mRNA, microRNA, long non-coding RNA are emerging as a biomarker, and considered to more accurately assess classification, prediction, diagnosis, recurrence and response to disease therapy. Therefore, it is necessary to develop a detection method for such biomarkers as described above.

Currently, the most widely used RNA marker is the FISH analysis. However, fabrication of probes and treatment with various reagents are done at vast expense, and require very complicated experimental methods, thus being likely to fail without very high skilled researchers.

Further, the FISH analysis takes a long time, and thus is time-consuming to determine whether a patient has a disease by RNA marker detection when using tissue sections of the patient. Therefore, this method is an obstacle to rapid diagnosis of a disease and to establishing treatment policy of the disease.

Accordingly, there is a requirement for developing a novel disease diagnostic technique based on nanotechnology involving simple experimental procedures as well as a simple probe with relatively low costs.

### SUMMARY

Accordingly, an object of the present invention is to provide a graphene nanosensor for detecting a target RNA, capable of substituting a conventional FISH analysis and being diversely applicable to the entire basic biological fields.

Another object of the present invention is to provide a graphene nanosensor for detecting a target RNA, capable of simply, quickly and accurately detecting RNA (mRNA, microRNA, long non-coding RNA) biomarkers with disease-specific over-expression and an expression level thereof in living tissues or cells.

In addition, another object of the present invention is to provide a graphene nanosensor for detecting a target RNA, capable of easily observing target RNA inhibitory effect during siRNA treatment.

Further, another object of the present invention is to provide a graphene nanosensor for detecting a target RNA, capable of obtaining a product with high reliability and resolution.

Further, another object of the present invention is to provide a graphene nanosensor for detecting a target RNA, capable of detecting different RNAs present in a single cell simultaneously.

Further, another object of the present invention is to provide a graphene nanosensor for detecting a target RNA, which can be utilized in all diseases possibly identified using tissues or cells without particular limitation to specific diseases.

Further, another object of the present invention is to provide a graphene nanosensor for detecting a target RNA, enabling rapid diagnosis of a disease and helpful for establishing treatment policy of the disease.

Further, another object of the present invention is to provide a graphene nanosensor for detecting a target RNA, capable of detecting RNA (mRNA, microRNA, long non-coding RNA) biomarkers with low costs.

Furthermore, another object of the present invention is to provide a graphene nanosensor for detecting a reduced target RNA having high dispersion properties.

(1) A graphene nanosensor for detecting a target RNA, including a nanosensor which is fabricated by contacting a graphene oxide, which is under pegylation and has a nuclear localization sequence attached thereto, to a peptide nucleic acid (PNA) aptamer coupled to a target RNA and labeled with a fluorescent dye.

(2) The graphene nanosensor for detecting a target RNA according to the above (1), wherein the fluorescent dye is at least one selected from the group consisting of fluorescein amidite (FAM) and hexachloro-fluorescein (HEX).

(3) The graphene nanosensor for dementia diagnosis according to the above (1), wherein the PNA probe is labeled with a plurality of fluorescent dyes.

(4) The graphene nanosensor for detecting a target RNA according to the above (1), wherein the graphene oxide is a reduced graphene oxide.

(5) The graphene nanosensor for detecting a target RNA according to the above (1), wherein the graphene oxide has a thickness of 30 to 120 nm.

(6) The graphene nanosensor for detecting a target RNA according to the above (1), wherein the nuclear localization sequence includes at least one selected from the group consisting of PKKKRKV (SEQ ID NO: 4), KR[PAATKKAGQA]KKKK (SEQ ID NO: 5), AVKRPAATKKAGQAKKKKLD (SEQ ID NO: 6), MSRRRKANPTKLSENAKKLAKEVEN (SEQ ID NO: 7), PAAKRVKLD (SEQ ID NO: 8), KLKIKRPVK (SEQ ID NO: 9), GRKKRRQRRRPQ (SEQ ID NO: 10) and KIPIK (SEQ ID NO: 11).

(7) The graphene nanosensor for detecting a target RNA according to the above (1), wherein the PNA aptamer labeled with the fluorescent dye is added in an amount of 30 pmol to 50 pmol.

(8) The graphene nanosensor for detecting a target RNA according to the above (1), wherein the graphene oxide is added in an amount of 0.3 μg to 0.5 μg.

(9) The graphene nanosensor for detecting a target RNA according to the above (1), wherein the detection of a target RNA is performed in at least one selected from a group consisting paraffin tissues, frozen tissues and cells.

(10) The graphene nanosensor for detecting a target RNA according to the above (9), wherein the tissue is a tissue section.

The graphene nanosensor for detecting a target RNA according to the present invention may replace the conventional FISH analysis method, thus being diversely applied to the entire basic biological fields.

The graphene nanosensor for detecting a target RNA according to the present invention may simply, quickly and accurately detect RNA (mRNA, microRNA, long non-coding RNA) biomarkers that have disease-specific over-expression, as well as an expression level thereof, in living tissues or cells.

The graphene nanosensor for detecting a target RNA according to the present invention may easily and quickly identify whether a specific RNA of interest is expressed or not throughout the embryonic tissues and a distribution thereof during histogenesis.

The graphene nanosensor for detecting a target RNA according to the present invention may easily observe target RNA inhibitory effects during siRNA treatment.

The graphene nanosensor for detecting a target RNA according to the present invention may obtain a product with high reliability and resolution.

The graphene nanosensor for detecting a target RNA according to the present invention may enable complex detection of multiple target RNAs thus to detect different RNAs present in a single cell simultaneously.

The graphene nanosensor for detecting a target RNA according to the present invention may detect RNA (mRNA, microRNA, long non-coding RNA) biomarkers with low costs.

The graphene nanosensor for detecting a target RNA according to the present invention may be utilized in all diseases possibly identified using tissues or cells without particular limitation to specific diseases in an aspect of clinical application.

The graphene nanosensor for detecting a target RNA according to the present invention may identify whether or not to suffer from any disease, detail types and/or severity of the disease, etc.

The graphene nanosensor for detecting a target RNA according to the present invention may desirably select or screen patient-specific medicines.

The graphene nanosensor for detecting a target RNA according to the present invention may enable rapid diagnosis of a disease and be helpful for establishing treatment policy of the disease.

### DESCRIPTION OF THE DRAWINGS

FIG. 1 is a schematic view illustrating one embodiment of the present invention.

FIG. 2 schematically illustrates major points of procedures for graphene in situ hybridization.

FIG. 3 is a schematic view illustrating an experimental method of a graphene sensor (GISH).

FIG. 4 illustrates visualization of changes in BC1 RNA (Brain Cytoplasmic RNA1) expression, which shows tile scan microscope images obtained from formalin-fixed paraffin embedded (FFPE) brain tissues in the entire coronal portion.

FIG. 5 illustrates observed images of BC1 distributed in cells along the hippocampus and the cerebral cortex layer at high magnification through FAM, wherein the scale bar is 200 μm in the left panel and 20 μm in the right panel.

FIG. 6 illustrates strong FAM signals in BC1 RNAs distributed widely in the thalamus, cerebellum, cerebral cortex and olfactory bulb in the brain sagittal sections of a mouse.

FIG. 7 illustrates images of the whole mouse brain having CLARITY by a nerve transmission system, wherein (a) is visualization of three-dimensional (3D) immune histological data to demonstrate tyrosine hydroxylase (TH)-positive neurons and fibers in the mouse brain. The normal mouse brain was washed in 1^{st} week and treated with a primary antibody in 2^{nd} week. Then, the treated brain was washed in next 1^{st} week and stained with a secondary antibody in next 2^{nd} week, followed by obtaining images with 2500 μm in the abdominal part using 10× water-precipitated lens. D, V, A and P show the dorsal part, the abdomen, front and rear sides, respectively. Further, (b to d) show optical portions with different depths which correspond to dotted box areas in the upper, middle and lower portions of the above A part, respectively, clearly illustrating that TH-positive neurons have been well labeled, and could be seen even in 2,460 μm depth of the whole brain (CPu, caudate putamen; PO, preoptic nucleus; VTA, ventral tegmental area; SNR, substantia nigra; RR, retrorubral nucleus; DR, dorsal raphe. Scale bars, 700 μm (a) and 100 μm)

FIG. 8 illustrates BC1 expression of each of the normal mouse brain and the mouse brain suffering from Alzheimer's disease (AD) via FAM-PNABC1-GO. Brains of the five mutations, that is, 5×FAD AD mice (at the age of 2 to 8 months) were classified into a RNA grade. Tissues thereof were fixed with 4% formaldehyde and subjected to deparaphilization. Thereafter, each of the prepared tissue samples was treated with FAM-PNABC1-GO. Confocal laser microscopic image exhibited that a BC1 level is increased in CA3 hippocampus neurons of AD mouse having a dystrophic cell form. This demonstrates that a correlation between a high grade BC200 expression in the hippocampus region and clinical dementia conditions

FIG. 9 illustrates BC expression in each of the normal mouse brain and the mouse brain suffering from Alzheimer's disease through FAM-PNABC1-GO. It was represented that a distribution of BC1 levels in dentate gyrus of 5×FAD mouse becomes hetereogeneous as fast as that of the mouse at the age of two months, and showed strong expression along the subgranular area where neural stem cells are present. Positive cells with increased BC1 are present around an intergranular layer, which mainly appears in amorphous morphological characteristics represent in a myriad of modified dystrophic cells. The mouse having mutation of five genes, which tend to be 5×FAD AD-similar phenotype, exhibited a deterioration in spatial memory in a Y-maze test.

FIG. 10 illustrates results of comparison between a fluorescent signal after treatment with FAM-linked PNA21-GO and a fluorescent signal after blocking FAM-PNA21-GO with a competitive inhibitor.

FIG. 11 illustrates results of GISH experiments performed on miR-124 having neuron-specific expression as a target.

FIG. 12 illustrates observed results of graphene sensor related experiments performed on miR-21 having breast cancer cell-specific expression as a target, in particular, the stronger fluorescent signal observed when four (4) fluorescent dyes are coupled to a miR-21 probe and treated in order to amplify the fluorescent signal.

FIG. 13 illustrates results of experiments for assessing expression levels of miR-21 in tissue sections from a patient having brain tumor by means of a graphene sensor.

FIG. 14 illustrates an example wherein PNA concentration is fixed while increasing a graphene concentration in order to determine optimal concentrations of the graphene and PNA probe, in particular, observed results of fluorescence recovery by treating the determined PNA-GO sensor with a target BC1 RNA oligomer.

FIG. 15 illustrates results of study for assessment of BC1 expression levels for each tissue.

FIG. 16 illustrates results of treatment of the BC1 targeting graphene sensor in brain tissues over time.

FIG. 17 illustrates results of study for identification of correlation between the PCR result and the graphene sensor result in response to BC1 expression.

FIG. 18 illustrates identified results of improvement in sensitivity of the graphene sensor, as compared to the conventional method, that is, FISH.

FIG. 20 illustrates identified results of abnormal response for 8 months by a rat with dementia through Y-maze behavior analysis.

FIG. 21 illustrates observed results of abnormal cell shapes in the brain of the rat with dementia through H & E results.

FIG. 22 illustrates results of identifying that any fluorescent signal is not exhibited when making a scramble form of a probe and treating brain tissues with the same in order to verify specificity of the graphene sensor to the target miRNA.

FIG. 23 illustrates results of identifying that a signal is reduced when treating PNA-GO not binding with the fluorescent dye and blocking the same.

FIG. 24 illustrates observed results of miR-21 expression in brain tissues of a normal human and a patient with brain tumors, respectively.

### DETAILED DESCRIPTION

The present invention relates to a graphene nanosensor for detecting a target RNA, including a nanosensor which is fabricated by contacting a graphene oxide, which is under pegylation and has a nuclear localization sequence attached thereto, to a peptide nucleic acid (PNA) aptamer coupled to a target RNA and labeled with a fluorescent dye, thereby accomplishing the following advantages, including: replacing the conventional FISH analysis method, thus being diversely applied to the entire basic biological fields; simply, quickly and accurately detecting RNA biomarkers that have disease-specific over-expression, as well as an expression level thereof, in living tissues or cells; easily and quickly identifying whether a specific RNA of interest is expressed or not throughout the embryonic tissues and a distribution thereof during histogenesis; easily observing target RNA inhibitory effects during siRNA treatment; enabling RNA detection even in living tissues thus to trace changes in RNA markers; obtaining a product with high reliability and resolution; enabling complex detection of multiple target RNAs thus to detect different RNAs present in a single cell simultaneously; being utilized in all diseases possibly identified using tissues or cells without particular limitation to specific diseases in an aspect of clinical application; identifying whether or not to suffer from any disease, details and/or severity of the disease, etc.; desirably selecting or screening patient-specific medicines; in addition, enabling rapid diagnosis of a disease and being helpful for establishing treatment policy of the disease.

The present invention may rapidly and simply detect a target RNS with high sensitivity at low costs, thereby expecting superior effects when used in clinical applications and thus replacing the FISH method.

Hereinafter, the present invention will be described in detail.

The graphene nanosensor for detecting a target RNA according to the present invention may include a graphene nanosensor fabricated by contacting a graphene oxide, which is under pegylation and has a nuclear localization sequence attached thereto, to a peptide nucleic acid (PNA) aptamer coupled to a target RNA and labeled with a fluorescent dye.

The target RNA in the present invention may be expressed in a single-strand form or may include mRNA or other RNA present in a single or double-strand in adult animal cells.

The target RNA may include, for example, BC1 RNA (BCYRN1, brain cytoplasmic RNA 1). In such a case, the graphene nanosensor of the present invention may diagnose dementia.

If the target RNA is BC1 RNA (BCYRN1, brain cytoplasmic RNA1), it may include a sequence of a specific subject that requires detection of BC1 RNA sequence of the graphene nanosensor according to the present invention. For instance, a human may have a nucleotide sequence of SEQ ID NO. 1 (ggccgggcgcggtggctcacgcctgtaatcccagctctcagggaggctaagaggcgggaggatagcttgagcccagga gttcgagacctgcctgggcaatatagcgagaccccgttctccagaaaaaggaaaaaaaaaaacaaaagacaaaaaaaaaa taagcgtaacttccctcaaagcaacaaccccccccccccttt, NCBI Reference Sequence NR_001568.1), but it is not limited thereto.

In the context of the present invention, coupling to the target RNA refers to complementary binding with the mRNA or other RNAs expressed or present in the adult animal cells, however, the coupling does not have to be the 100% complementary binding with the mRNA or other RNAs.

The aptamer in the present invention refers to a single-stranded nucleic acid (DNA, RNA or modified nucleic acid) having a stable three-dimensional (3D) structure in itself and characteristics capable of binding with a target molecule such as metal ions, protein, cells, with high affinity and specificity, wherein this aptamer can be obtained by isolating an oligomer coupled to any specific chemical molecule or biological molecule with high affinity and selectivity according to an advanced method using an oligonucleotide library called SELEX (systematic evolution of ligands by exponential enrichment).

Similar to the DNA or RNA, a peptide nucleic acid (PNA) in the present invention refers to an artificially synthesized polymer.

In the present invention, the PNA may be more stable to a stimulus such as heat or pH than the DNA or RNA while having similar features to the same. Further, this is not easily recognized by a nucleic acid degrading enzyme (‘nuclease’) and thus may be stable against a degradation by the enzyme.

The PNA aptamer according to the present invention may have a stable 3D structure and be coupled to a target molecule with high affinity and specificity.

According to one embodiment of the present invention, the PNA aptamer may be formed by modifying a RNA probe into PNA, thereby reducing enzyme-related degradation and detecting the target RNA with higher sensitivity.

The fluorescent dye in the present invention is a fluorescence-emitting material to absorb an energy at a specific wavelength and then emit again the energy at another specific wavelength.

According to one embodiment of the present invention, the fluorescent dye may undergo fluorescence quenching through energy transfer when this is labeled on the PNA aptamer and close to graphene. The PNA aptamer may be a PNA probe, but it is not limited thereto.

The fluorescent dye in the present invention is not particularly limited in an aspect of types thereof, but may include any typical fluorescent dye known in the art so long as the fluorescence can be quenched through fluorescence resonance energy transfer (RET).

According to one embodiment of the present invention, the fluorescent dye may include at least one selected from the group consisting of fluorescein amidite (FAM), hexachloro-fluorescein (HEX), NEDTN and Texas Red™.

The PNA aptamer labeled with a fluorescent dye may be coupled to a carbon ring in a graphene oxide through pi-pi interaction, however, it is not limited to this theory.

As necessary, the PNA aptamer may be labeled with multiple fluorescent dyes to amplify a fluorescence signal, thereby more improving the sensitivity. For instance, two, three, four or more fluorescent dyes may be labeled on the PNA aptamer, but it is not limited thereto.

The graphene may be synthesized by peeling off graphite having a layered structure through mechanical or ultrasonic treatment or by means of epitaxial growth, chemical vapor deposition (CVD), etc., and may have a two-dimensional sheet shape including multiple layers, as well as structural and/or electrical properties (electrical conductivity) similar to those of graphite.

The graphite oxide synthesized by oxidation of graphite may have a layered structure similar to graphite, wherein an oxygen containing functional group such as epoxy, hydroxyl, carboxyl, etc. is present in the interlayer of the surface of a layer thus to have an interlayer distance of about 0.7 nm or more, which is wider than that of the graphite (0.34 nm). Such an increase in the interlayer distance may enable the layer to be easily peeled off due to an electrostatic repulsion between oxygens in the interlayer, and such peeled off graphite oxide is defined as a graphene oxide.

In the present invention, graphene synthesized by removing oxygen from the graphene oxide is defined as a reduced graphene oxide.

According to one embodiment of the present invention, the graphene oxide may be a reduced graphene oxide.

Pegylation stated in the present invention means chemically linking to polyethylene glycol (PEG), and when the graphene oxide is under pegylation according to the present invention, different effects such as a decrease of immunogenicity, increase of solubility, increase of dispersibility in a physiological active solution, improvement of stability, increase of sensitivity for detection of target RNA, or the like, may be achieved.

A nuclear localization sequence (NLS) in the present invention may be an amino acid sequence which is a tag protein to transport a material to a nucleus of a cell. When the NLS is attached to the graphene oxide according to the present invention, the graphene nanosensor can be introduced into the nucleus thus to enable detection of a target RNA in tissues or cells with high resolution and reliability.

In the present invention, types of NLS are not limited so long as the NLS can be attached to the graphene and enable the graphene nanosensor to be introduced into the nucleus, and any NLS known in the art may be used.

According to one embodiment of the present invention, the NLS may include at least one selected from the group consisting of PKKKRKV (SEQ ID NO: 4), KR[PAATKKAGQA]KKKK (SEQ ID NO: 5), AVKRPAATKKAGQAKKKKLD (SEQ ID NO: 6), MSRRRKANPTKLSENAKKLAKEVEN (SEQ ID NO: 7), PAAKRVKLD (SEQ ID NO: 8), KLKIKRPVK (SEQ ID NO: 9), GRKKRRQRRRPQ (SEQ ID NO: 10) and KIPIK (SEQ ID NO: 11).

In the present invention, PKKKRKV (SEQ ID NO: 4) is a first NLS found in an SV40 Large T-antigen (a monopartite NLS).

KR[PAATKKAGQA]KKKK (SEQ ID NO: 5) and AVKRPAATKKAGQAKKKKLD (SEQ ID NO: 6) are NLSs of a nucleoplasmin (a protein that participates in condensation of chromosomes), MSRRRKANPTKLSENAKKLAKEVEN (SEQ ID NO: 7) is a NLS of EGL-13, PAAKRVKLD (SEQ ID NO: 8) is a NLS of c-Myc, and KLKIKRPVK (SEQ ID NO: 9) is a NLS of TUS-protein.

Further, by attaching the NLS to the graphene oxide subjected to pegylation according to the present invention, a target RNA may be detected in the tissue or cell level, with high reliability and resolution.

The graphene oxide according to the present invention may have a thickness of 30 nm to 120 nm, for example, 50 nm to 100 nm.

In the present invention, the target RNA means a RNA which can be coupled with the PNA aptamer, and it is not limited so long as it is mRNA or other RNAs present in the tissue or cell.

According to one embodiment of the present invention, the PNA aptamer labeled with a fluorescent dye to quench fluorescence may be added in an amount of 30 pmol to 50 pmol. For instance, the PNA aptamer labeled with a fluorescent dye may be added in an amount of 40 pmol.

According to one embodiment of the present invention, graphene may be added in an amount of 0.3 μg to 0.5 μg in order to quench fluorescence. For instance, graphene may be added in an amount of 0.4 μg.

In the present invention, detection of a target RNA may be performed in at least one selected from the group consisting of paraffin tissues, frozen tissues and cells. The tissue may be tissue sections.

The paraffin tissue in the present invention may be obtained by penetrating paraffin into a tissue and solidifying the same.

When performing the detection of a target RNA in paraffin tissues, frozen tissues or cells according to the present invention, a change in the target RNA may be traced thus to easily diagnose a disease or determine the progress of the disease. Further, it is possible to implement personalized therapy through a change in the target RNA according to treatment of the disease.

Hereinafter, the following examples will be proposed to facilitate understanding of the present invention. However, these examples are given for more easily understanding the present invention only, and the present invention is not construed to be limited to the following examples.

### EXAMPLE

Experimental Material and Method

Synthesis of Fluorescent Probe

A probe coupled to a target RNA was synthesized in a peptide nucleic acid (PNA) form by Panagene Inc. (Republic of Korea) in order to increase chemical and biological stabilities. The synthesized probe sequence was GGTCTTTTTGTTATTTTGTCTT (FAM-PNA-BC1-1, SEQ ID NO: 2) and TTCTGTTTTATTGTTTTTCTGG (FAM-PNA-scr1, scramble, SEQ ID NO: 3). All probes were synthesized in a form in which an O-linker spacer is added to an N-terminal and then a fluorescent dye, FAM, is linked to the same.

Synthesis of Graphene Oxide (GO)

The graphene oxide was prepared so as to have a uniform size of 50 to 100 nm through screening.

Quenching of Fluorescent Probe

Conditions for quenching a fluorescent probe are as follows: after adding 40 pmol of fluorescent probe as well as 0.4 μg of graphene oxide and pouring 0.01M Tris-HCl buffer (pH 7.4) to prepare a solution in a total volume of 100 μl, the solution was subjected to a reaction at room temperature for 10 minutes, followed by measurement of fluorescence using a fluorometer.

Assessment of Fluorescence in Mouse Brain Tissue

A coronal or sagittal section was prepared from brain tissues of a normal mouse, followed by completing a paraffin block. After slicing the block in a thickness of 4 μm, the section was adhered to a slide to prepare the tissue. After removing the wax at the cut section of the prepared tissue with xylene, ethanol at different concentrations was added thereto to hydrate the same. After washing the cut section of the tissue, the tissue was blocked with 5% BSA (in TBS) at room temperature for 1 hour. 40 pmol of fluorescent probe as well as 0.4 μg of graphene oxide were added and then 0.01M Tris-HCl buffer (pH 7.4) was poured to prepare a solution in a total volume of 100 μl. Then, the solution was subjected to a reaction at room temperature for 10 minutes. After reacting a fluorescent probe-graphene oxide composite in the tissue at 4° C. for 15 hours, the reaction product was washed. The tissue was subjected to counter staining using DAPI. A final result of the tissue was observed under a fluorescence microscope (Leica TCS SP8, Leica).

Experimental Result

Identification of Fluorescence Quenching by Graphene Oxide

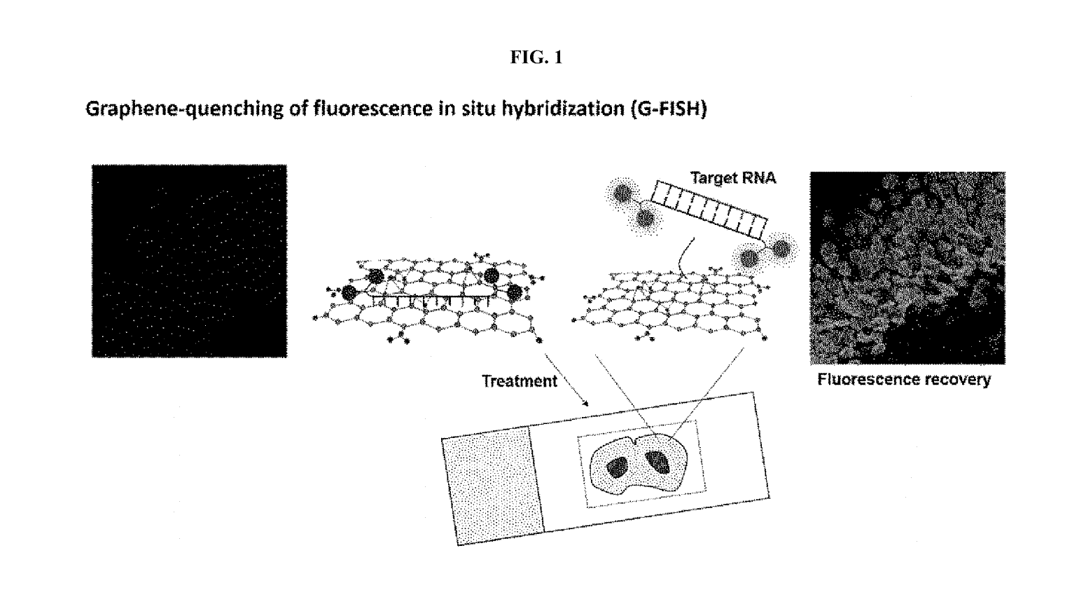

Whether fluorescence was quenched by graphene oxide or not was examined. As shown in FIG. 1, it could be understood that, compared to a fluorescence level of the fluorescent probe only (FAM-PNA only), the fluorescence level of the fluorescent probe-graphene oxide composite (AM-PNA-GO) was quenched by about 95%, which was substantially identical to a target probe (FAM-PNA-BC1-1) and a scramble probe (FAM-PNA-scr1).

Detection of Target RNA in Brain Tissue by Fluorescent Probe-Graphene Oxide Composite

A target RNA to be detected was RNA over-expressing in the mouse brain tissue, and a probe used for detection was synthesized to have an antisense sequence complementarily binding with the target RNA. The fluorescent probe is coupled to graphene oxide and enters into the tissue in a fluorescence quenching state, and when coming to the target RNA within the tissue, the fluorescent probe falls out of the graphene oxide and then is coupled to the target RNA, thereby enabling observation of fluorescence.

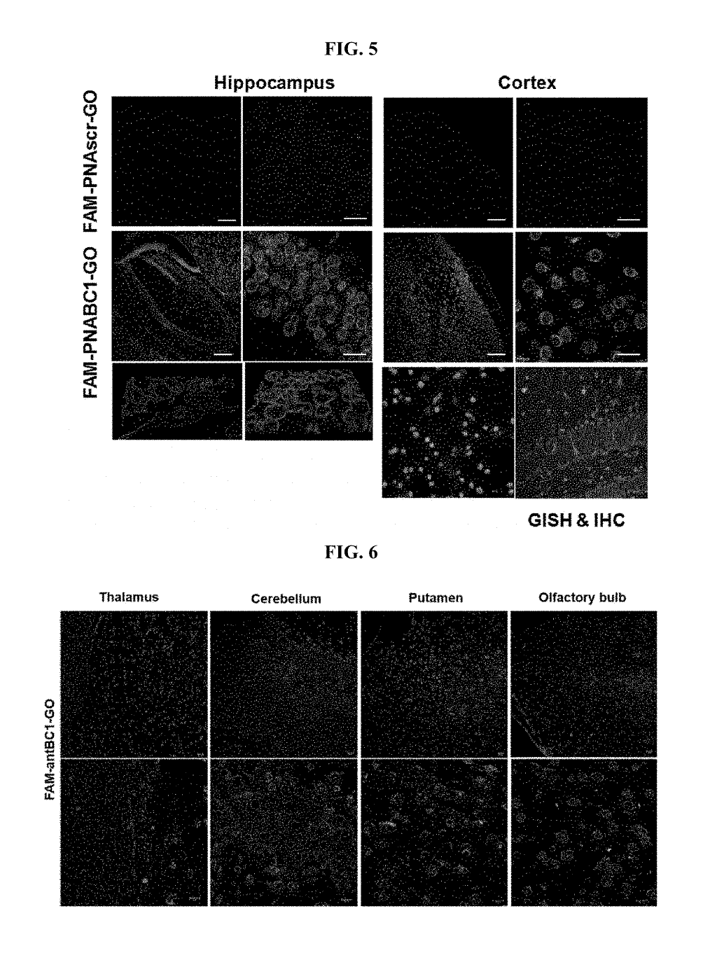

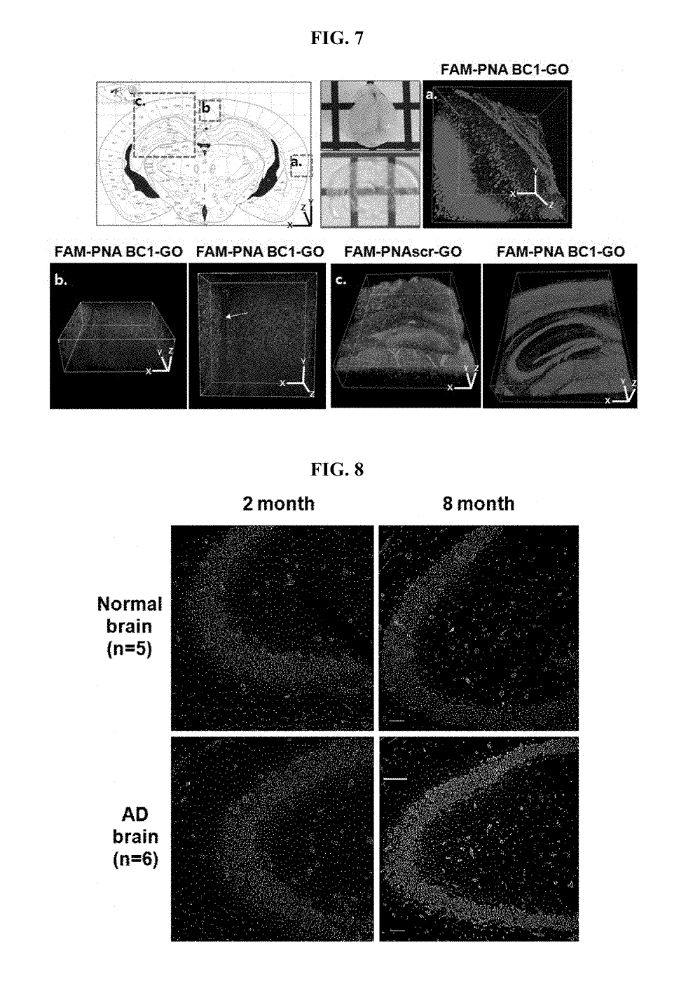

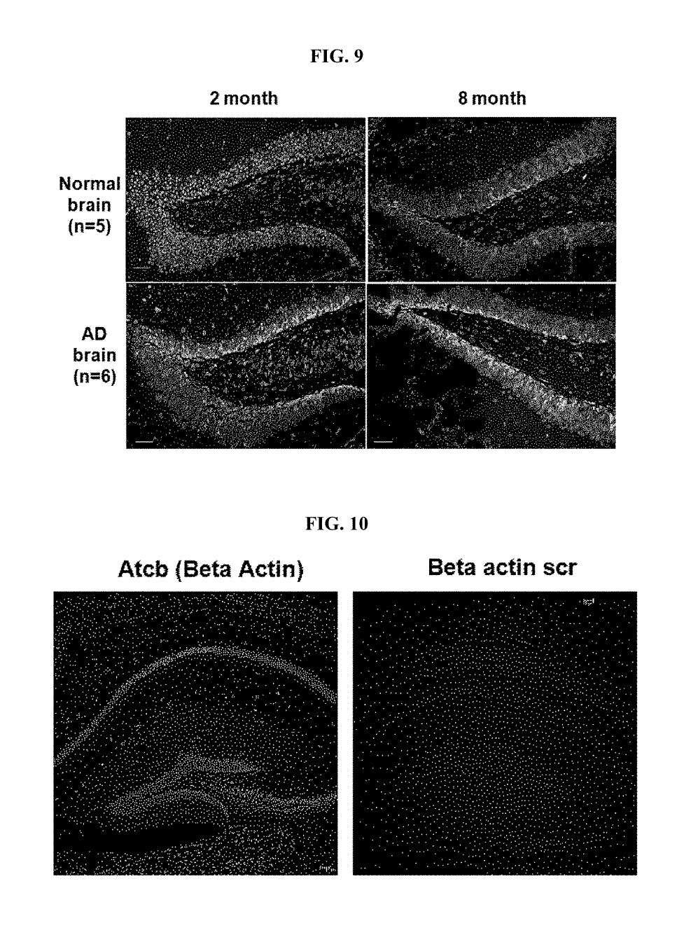

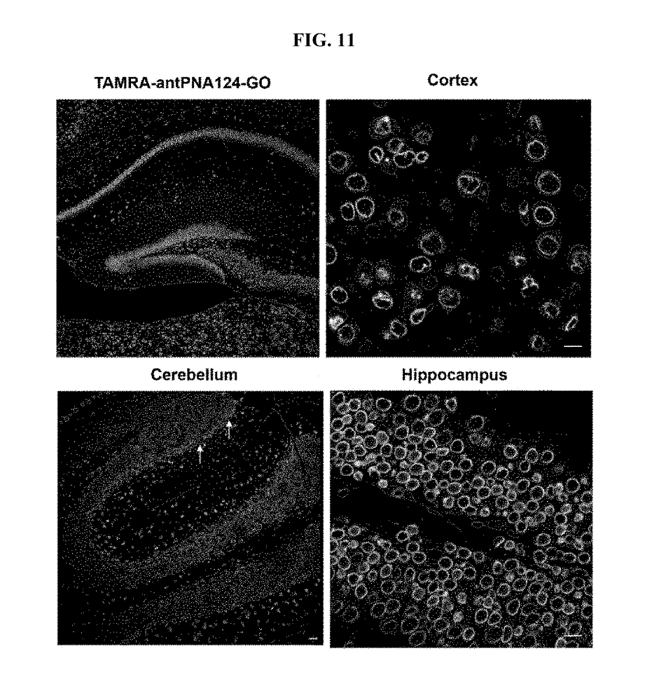

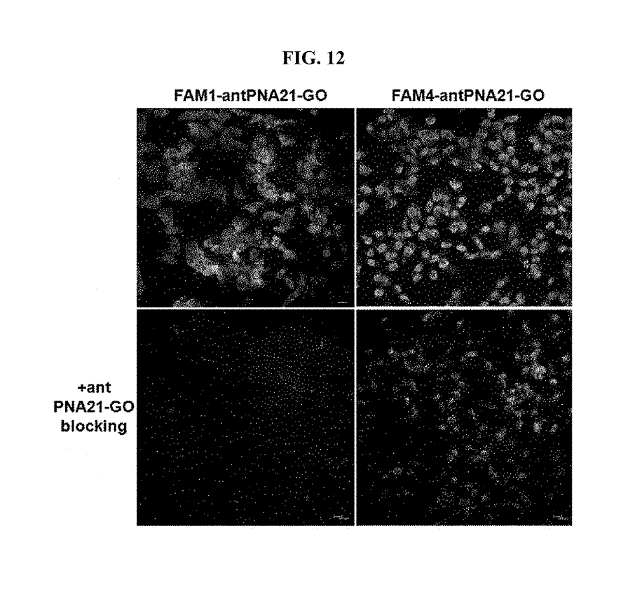

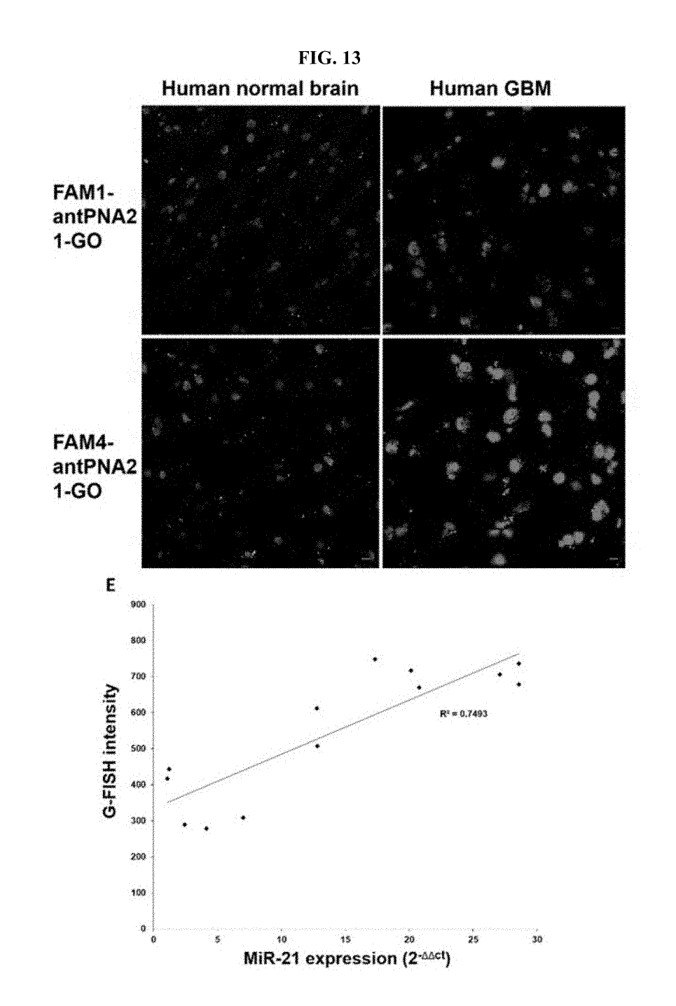

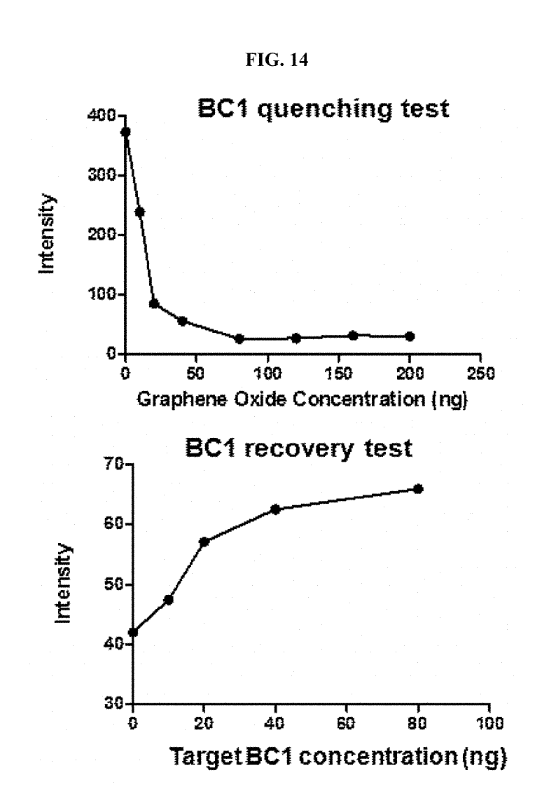

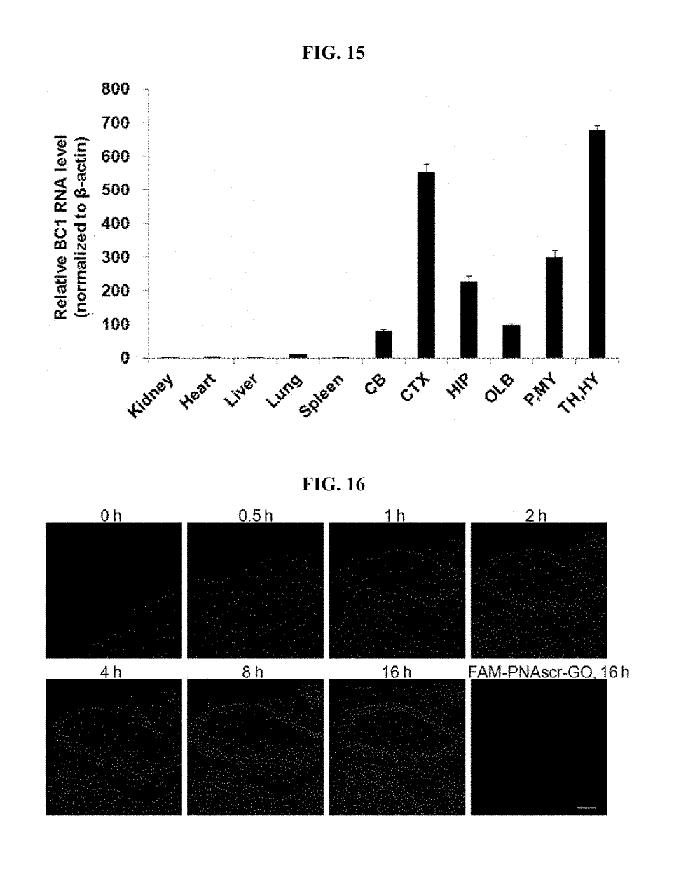

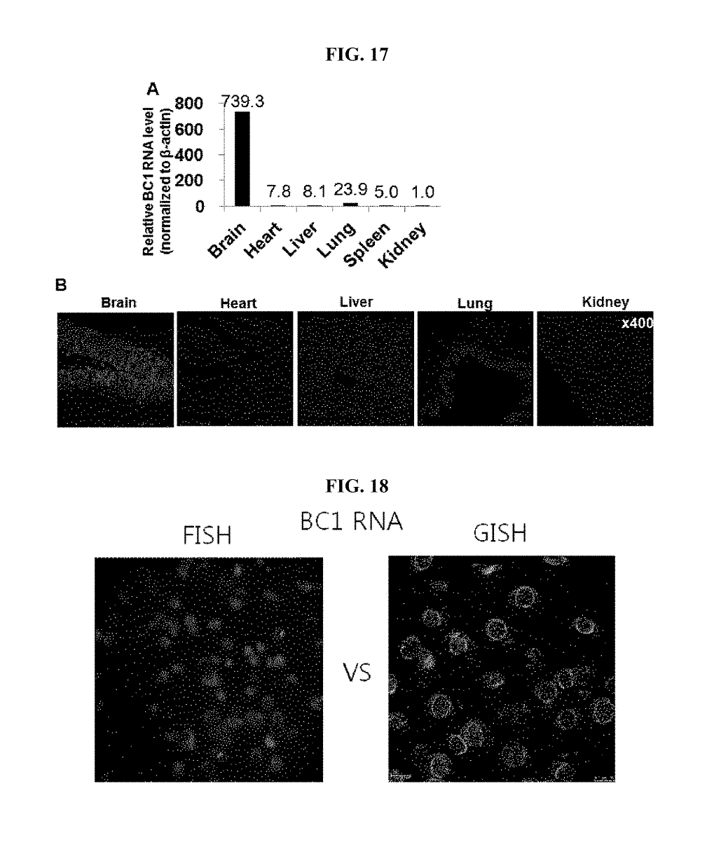

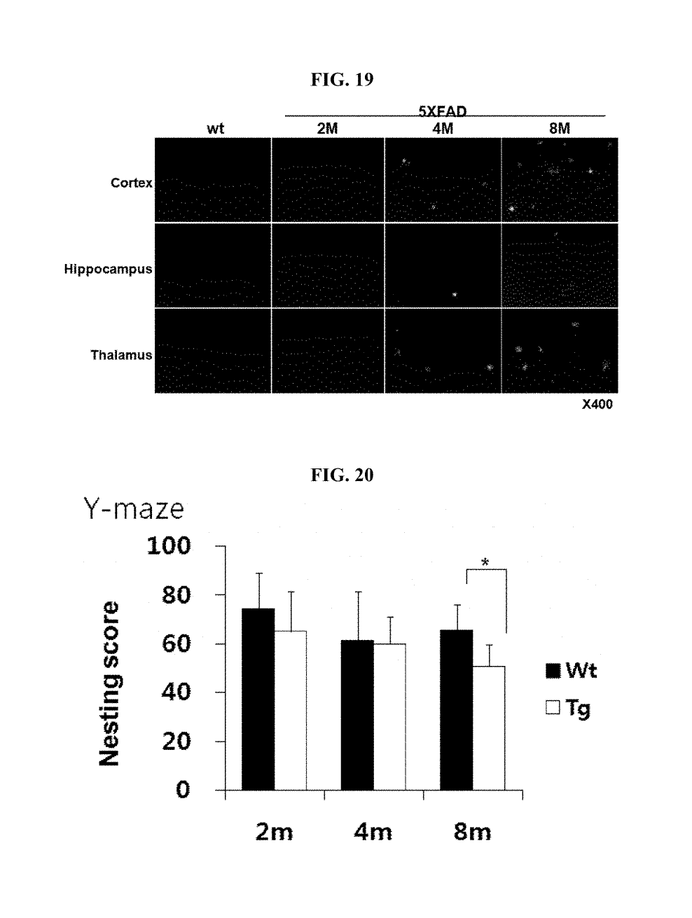

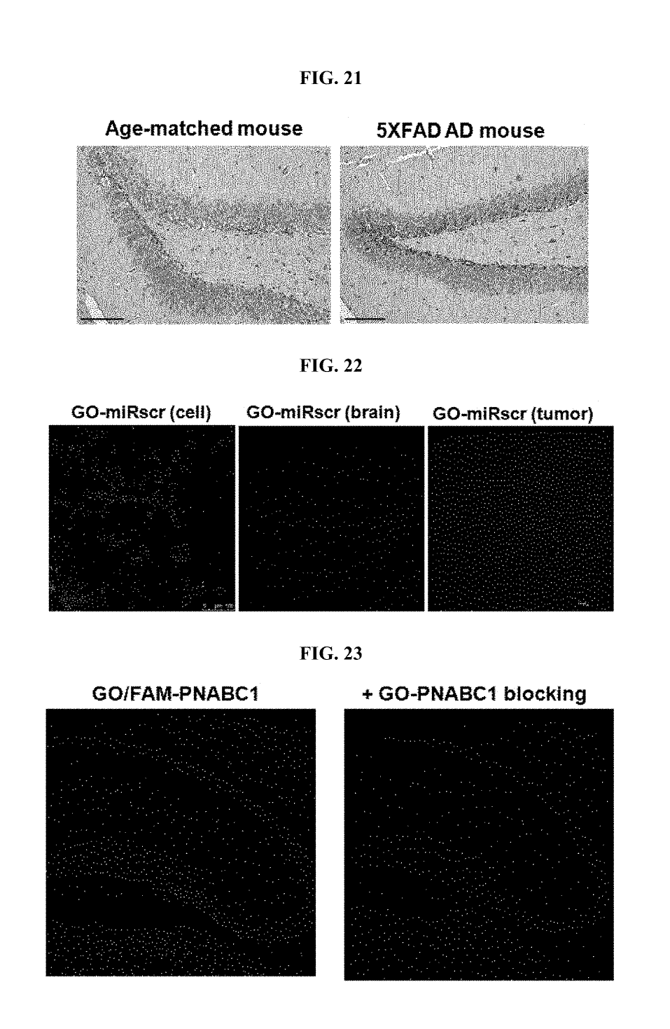

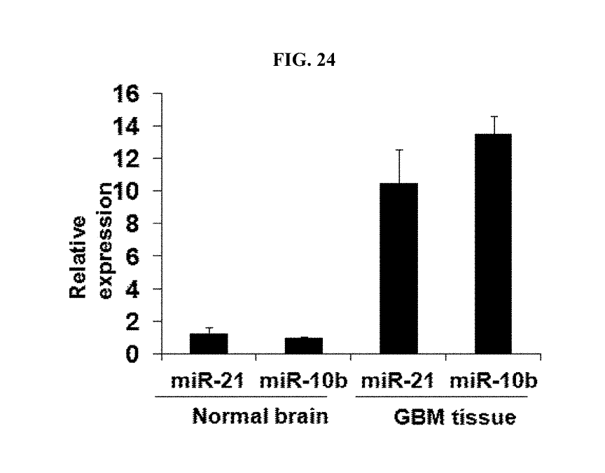

In order to examine whether the target probe can detect the target RNA over-expressing in the mouse brain tissue or not, the brain tissue of a normal mouse was removed and processed to prepare a tissue slide having a coronal section. When using FAM-PNA-BC1-1 as a target probe, expression of fluorescence was obviously detected in all of cortex (FIG. 5), hippocampus (FIG. 5) and thalamus (FIG. 6). On the other hand, when using FAM-PNA-scr1 as a scramble probe, there was no observation of fluorescence in any tissue (FIGS. 5 and 6).

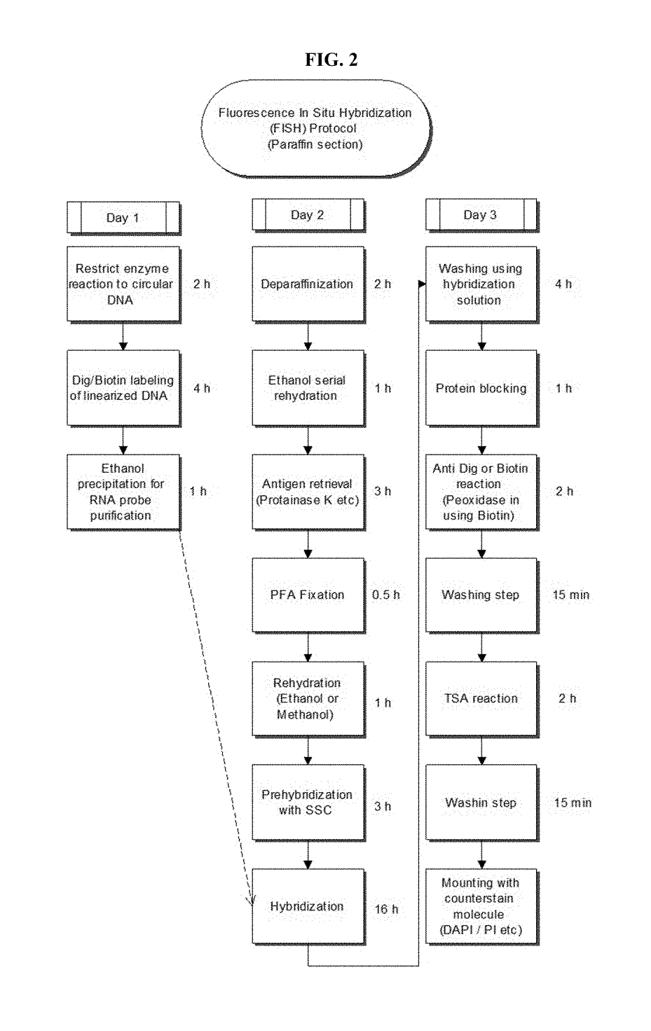

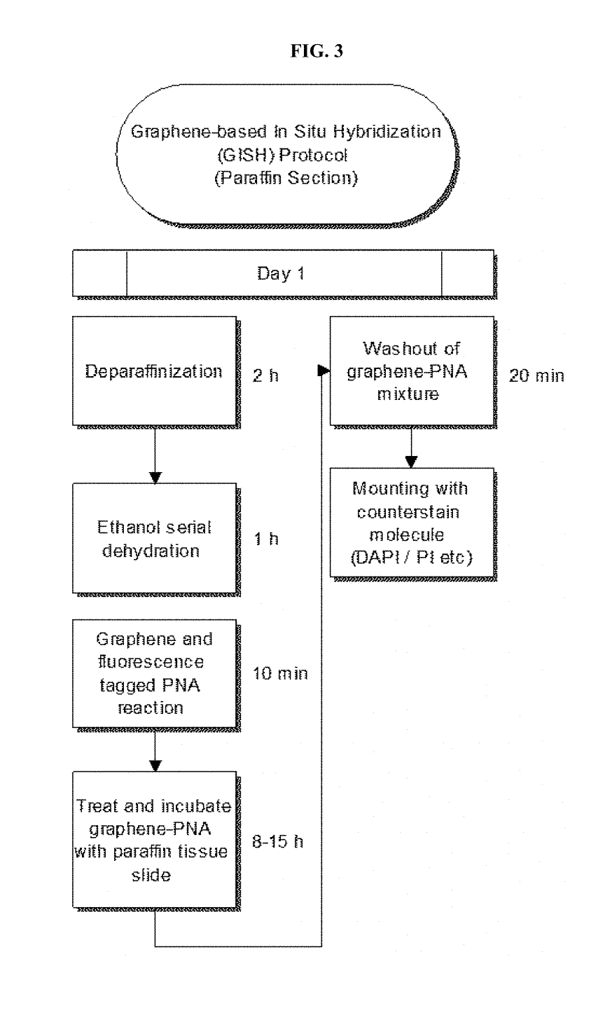

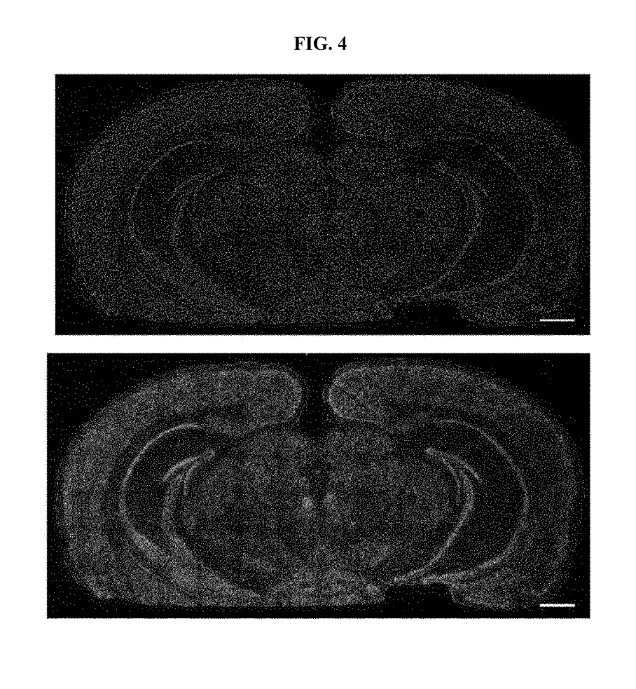

Among the results shown in FIG. 5, it was intended to investigate expression in a single cell by observing the cortex and hippocampus regions at a high magnification. As a result, expression of fluorescence was observed in the cytoplasms in all of cortex, hippocampus CA1, hippocampus CA3 and hippocampus DG. However, for FAM-PNA-scr1, fluorescence was not detected (FIG. 2).

In addition to the coronal section parts, in order to examine whether the target RNA can be detected in other brain regions, tissue slides having sagittal sections were prepared by separating the brain tissues of a normal mouse. When using FAM-PNABC1-1 as a target probe, expression of fluorescence was detected in all of midbrain, cerebellum, pons, putamen and olfactory bulb as well as cortex, hippocampus and thalamus which could be seen in the coronal section. On the other hand, for FAM-PNA-scr1, there was no observation of fluorescence in any tissue.

From the above results, it can be understood that graphene oxide under pegylation does not agglomerate in a physiologically active solution thus to increase dipersibility, the RNA probe may be modified into a peptide form (PNA aptamer) thus to minimize enzyme-related degradation, and a target RNA in cells may be easily, simply and rapidly detected with high reliability and resolution.

1. A graphene nanosensor for dementia diagnosis, comprising:
a graphene oxide; and
a peptide nucleic acid (PNA) probe labeled with a fluorescent dye,
wherein the PNA probe complementarily binds with Brain Cytoplasmic RNA1 (BC1 RNA).

2. The graphene nanosensor for dementia diagnosis according to claim 1, wherein the fluorescent dye is at least one selected from the group consisting of fluorescein amidite (FAM) and hexachloro-fluorescein (HEX).

3. The graphene nanosensor for dementia diagnosis according to claim 1, wherein the PNA probe is labeled with a plurality of fluorescent dyes.

4. The graphene nanosensor for dementia diagnosis according to claim 1, wherein the graphene oxide is a reduced graphene oxide.

5. The graphene nanosensor for dementia diagnosis according to claim 1, wherein the graphene oxide has a thickness of 30 to 120 nm.

6. The graphene nanosensor for dementia diagnosis according to claim 1, wherein the PNA probe sequence includes GGTCTTTTTGTTATTTTGTCTT (SEQ. ID NO. 2).

7. The graphene nanosensor for dementia diagnosis according to claim 1, wherein the PNA prober labeled with the fluorescent dye is added in an amount of 30 pmol to 50 pmol.

8. The graphene nanosensor for dementia diagnosis according to claim 1, wherein the graphene oxide is added in an amount of 0.3 μg to 0.5 μg.

9. The graphene nanosensor for dementia diagnosis according to claim 1, wherein the diagnosis of dementia is performed in at least one selected from a group consisting paraffin tissues, frozen tissues and cells.

10. The graphene nanosensor for dementia diagnosis according to claim 9, wherein the tissue is a tissue section.


SEQUENCE LISTING (11 sequences)

SEQ ID NO: 1 - DNA, 200 bp, Homo sapiens
ggccgggcgcggtggctcacgcctgtaatcccagctctcagggaggctaagaggcgggaggatagcttgagcccaggagttcgagacctgcctgggcaatatagcgagaccccgttctccagaaaaaggaaaaaaaaaaacaaaagacaaaaaaaaaataagcgtaacttccctcaaagcaacaaccccccccccccttt

SEQ ID NO: 2 - DNA, 22 bp, Artificial Sequence
  PNA probe FAM-PNA-BC1-1
ggtctttttgttattttgtctt

SEQ ID NO: 3 - DNA, 22 bp, Artificial Sequence
  PNA probe FAM-PNA-scr1
ttctgttttattgtttttctgg

SEQ ID NO: 4 - protein, 7 aa, Artificial Sequence
  nuclear localization sequence
PKKKRKV

SEQ ID NO: 5 - protein, 16 aa, Artificial Sequence
  nuclear localization sequence
KRPAATKKAGQAKKKK

SEQ ID NO: 6 - protein, 20 aa, Artificial Sequence
  nuclear localization sequence
AVKRPAATKKAGQAKKKKLD

SEQ ID NO: 7 - protein, 25 aa, Artificial Sequence
  nuclear localization sequence
MSRRRKANPTKLSENAKKLAKEVEN

SEQ ID NO: 8 - protein, 9 aa, Artificial Sequence
  nuclear localization sequence
PAAKRVKLD

SEQ ID NO: 9 - protein, 9 aa, Artificial Sequence
  nuclear localization sequence
KLKIKRPVK

SEQ ID NO: 10 - protein, 12 aa, Artificial Sequence
  nuclear localization sequence
GRKKRRQRRRPQ

SEQ ID NO: 11 - protein, 5 aa, Artificial Sequence
  nuclear localization sequence
KIPIK